(19) United States
(12) Patent Application Publication
(10) Pub. No.: US 2015/0151090 A1
(43) Pub. Date: Jun. 4, 2015

(54) COAXIAL GUIDE COIL FOR INTERVENTIONAL CARDIOLOGY PROCEDURES

(71) Applicant: GMedix, Inc, Plymouth, MN (US)

(72) Inventors: Gregg Stuart Sutton, Maple Grove, MN (US); Jeffrey M. Welch, Maple Grove, MN (US); Karl V. Ganske, Hopkins, MN (US)

(21) Appl. No.: 14/406,465

(22) PCT Filed: Jun. 10, 2013
(86) PCT No.: PCT/US13/45045

§ 371 Date: Dec. 8, 2014

Related U.S. Application Data
(60) Provisional application No. 61/657,381, filed on Jun. 8, 2012.

(51) Int. Cl. A61M 25/09; A61B 17/12; A61F 2/95

(52) U.S. Cl. CPC A61M 25/09; A61F 2/95; A61B 17/12136; A61M 2025/09066; A61M 2025/09108; A61M 2025/09133; A61M 2025/09166; A61M 2025/09175; A61M 2025/0004

(57) ABSTRACT

The present application describes a coaxial guide coil catheter device to be placed inside of a standard cardiovascular guide catheter for facilitating extra support in the coronary artery when advancing interventional cardiology devices such as balloon catheters and stent delivery systems. This device is passed through the main lumen of a standard guide catheter so that the tip of the coaxial guide coil device is extended past the tip of the guide catheter and into the coronary artery. The proximal portion of the coaxial guide coil will remain within the guide catheter. The device improves the ability of the guide catheter to remain seated in the coronary ostium during interventional procedures.

This application is being filed as a PCT International Patent application on Jun. 10, 2013 in the name of NorMedix LLC, a U.S. national corporation, applicant for the designation of all countries and Gregg Stuart Sutton, a U.S. Citizen, Jeffrey M. Welch, a U.S. Citizen, and Karl V. Ganske, a U.S. Citizen, are inventors only for the designated states, and claims priority to U.S. Provisional Patent Application No. 61/657,381, filed Jun. 8, 2012, the contents of which are herein incorporated by reference in its entirety.

### FIELD OF THE INVENTION

The present invention relates to catheters. More specifically, the present invention relates to a coaxial guide coil catheter.

### BACKGROUND OF THE INVENTION

Current guide catheters have a propensity to back out of the coronary main branch or ostium when subsequent interventional coronary devices are passed through it and advanced more distally. This causes the operator to lose ability to advance coronary devices more distally.

Accordingly, there is a need for providing increased back-up support for guide catheters in interventional cardiology procedures.

### SUMMARY OF THE INVENTION

The present application describes a coaxial guide coil catheter device to be placed inside of a standard cardiovascular guide catheter for facilitating extra support in the coronary artery when advancing interventional cardiology devices such as balloon catheters and stent delivery systems. The device is passed through the main lumen of a standard guide catheter so that the tip of the coaxial guide coil device is extended past the tip of the guide catheter and into the coronary artery. The proximal portion of the coaxial guide coil can remain within the guide catheter. The device can improve the ability of the guide catheter to remain seated in the coronary ostium during interventional procedures.

The device, in certain embodiments, is made of a distal tip, a first polymer jacketed flexible coil section, an optional second flexible coil section (optionally unjacketed) and a push rod. The push rod is made, for example, of high tensile stainless steel and is attached to the main flexible coil/lumen section using suitable attachment techniques such as welding, brazing or soldering. The first polymer jacketed flexible coil section is desirably made with a jacket material of Pebax® or urethane, and the flexible coil is desirably made of stainless steel.

The optional second flexible coil section is made, for example, of stainless steel, has a diameter slightly larger than the first coil section diameter (1.03-1.1 times larger, for example) and has a proximal luminal entry point for device passage adjacent to the push rod attachment point. The coil structure in both the first and second flexible sections are preferably made of multiple filar windings, for example 8-12 filar windings, or can also me made using a single wire wind. The filar windings are typically rectangular, or substantially rectangular in cross section.

The outer diameter of both flexible sections is made to fit closely, but passably, within standard guide catheters. The distal tip portion of the device can be made of a soft polymeric material and is attached to the first flexible section. The distal tip section provides a soft, leading edge when inserted into the coronary vasculature. The tip portion optionally contains a radiopaque material which allows a physician or technician to visualize the tip on fluoroscopy during the procedure.

Upon placement of a standard guide catheter into the coronary main branch, a physician or technician can advance the device (coaxial guide coil) through the main lumen of the guide catheter using the push rod until the tip of the coaxial guide coil tip is extended past the guide catheter tip and further into the coronary artery. This distance can range, for example, from 5 to 25 cm depending on the situation.

The proximal portion of the coaxial guide coil can remain within the guide catheter. The physician or technician can then deliver the interventional therapeutic device such as a stent or balloon device over a guide wire through the main lumen of the guide catheter and through the lumen of the coaxial guide coil until the working portion of the interventional device is past the coaxial guide coil tip. The physician or technician can then treat the coronary lesion using standard technique, but will have added back-up support on the guide catheter to provide extra ability to push and advance the device.

The outside diameter of the second flexible coil section can be 1.1 times or less larger than the outside diameter of the first flexible coil section. The first flexible coil section can have an outside diameter of 0.05 inches or more. The first flexible coil section can have an outside diameter of 0.090 inches or less. In an embodiment, the outside diameter of the first flexible coil section can be 0.064 inches. The first flexible coil section can have an inside diameter of 0.047 inches or more. The first flexible coil section can have an inside diameter of 0.084 inches or less. In an embodiment the inside diameter of the first flexible coil section 320 can be 0.057 inches.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a side view of a coaxial guide coil catheter, according to an embodiment of the invention.

FIG. 2 is a bottom view of a coaxial guide coil catheter, according to an embodiment of the invention.

FIG. 3 is a side view of a coaxial guide coil catheter, according to an embodiment of the invention.

FIG. 4 is a cross-section view of a coaxial guide coil catheter shown in FIG. 3, according to an embodiment of the invention.

FIG. 5 is a view of a portion of FIG. 4, according to an embodiment of the invention.

FIG. 6 is a side view of a distal tip of a coaxial guide coil catheter, according to an embodiment of the invention.

FIG. 7 is a cross-section view of a coaxial guide coil catheter taken along line A-A in FIG. 2, according to an embodiment of the invention.

FIG. 8 is a view of the push rod connection with the collar, according to an embodiment of the invention.

FIG. 9 is a cross-section view of the connection between the push rod and the collar taken along line B-B in FIG. 2, according to an embodiment of the invention.

FIG. 10 is a cross-section view of a push rod taken along line C-C in FIG. 2, according to an embodiment.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present application describes a coaxial guide coil catheter device to be placed inside of a standard cardiovascular guide catheter for facilitating extra support in the coronary artery when advancing interventional cardiology devices such as balloon catheters and stent delivery systems. This device is passed through the main lumen of a standard guide catheter so that the tip of the coaxial guide coil device is extended past the tip of the guide catheter and into the coronary artery. The proximal portion of the coaxial guide coil can remain within the guide catheter. The device can improve the ability of the guide catheter to remain seated in the coronary ostium during interventional procedures.

The device, in certain embodiments, is made of a distal tip, a first polymer jacketed flexible coil section, an optional second flexible coil section (unjacketed) and a push rod. The push rod is made, for example, of high tensile stainless steel and is attached to the main flexible coil/lumen section using suitable attachment techniques such as welding, brazing or soldering. The first polymer jacketed flexible coil section is preferably made with a jacket material of Pebax® or urethane, and the flexible coil is typically made of stainless steel.

The optional second flexible coil section is also made, for example, of stainless steel, and can optionally have a diameter slightly larger than the first coil section diameter (1.03-1.1 times larger, for example) and has a proximal luminal entry point for device passage adjacent to the push rod attachment point. The coil structure in both the first and second flexible sections are desirably made of multiple filar windings for example 8-12 filar windings, or can also me made using a single wire wind.

The outer diameter of both flexible sections is made to fit closely, but passably, within standard guide catheters. The distal tip portion of the device can be made of a soft polymeric material and is attached to the first flexible section. The distal tip section provides a soft, leading edge when inserted into the coronary vasculature. The tip portion preferably contains a radiopaque material which allows the physician to visualize the tip on fluoroscopy during the procedure.

Upon placement of a standard guide catheter into the coronary main branch, the physician can advance the device (coaxial guide coil) through the main lumen of the guide catheter using the push rod until the tip of the coaxial guide coil tip is extended past the guide catheter tip and further into the coronary artery. This distance can range, for example, from 5 to 25 cm depending on the situation.

The proximal portion of the coaxial guide coil can remain within the guide catheter. The physician can then deliver the interventional therapeutic device such as a stent/balloon device over a guide wire through the main lumen of the guide catheter and through the lumen of the coaxial guide coil until the working portion of the interventional device is past the coaxial guide coil tip. The physician can then treat the coronary lesion using standard technique, but will have added back-up support on the guide catheter to provide extra ability to push and advance the device.

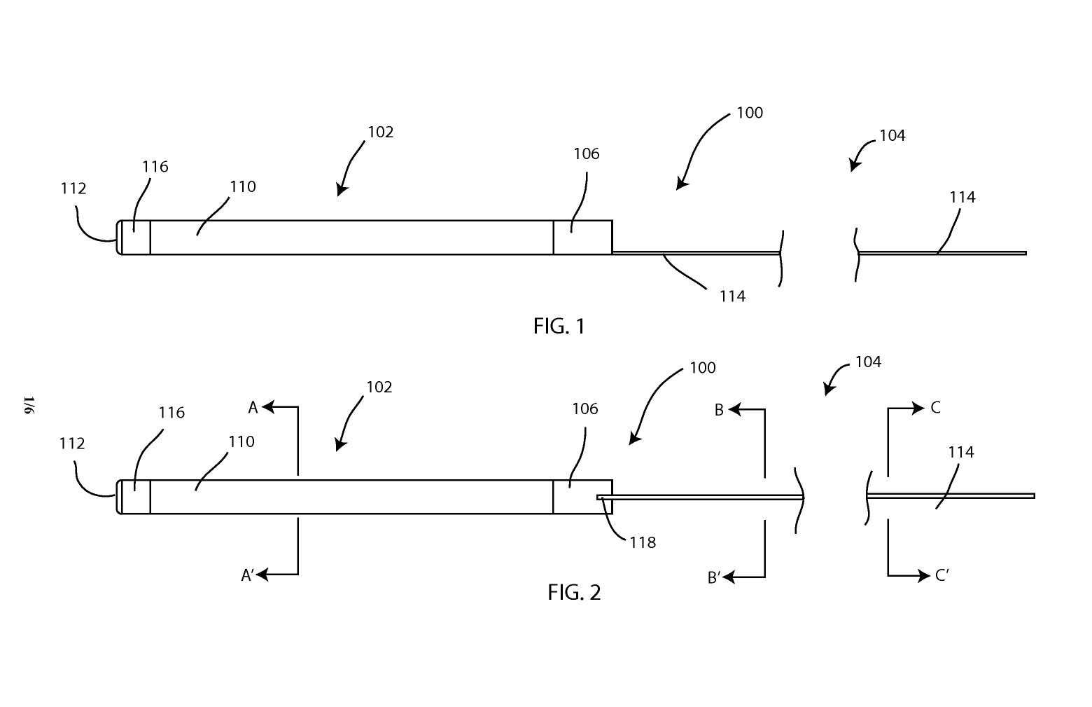

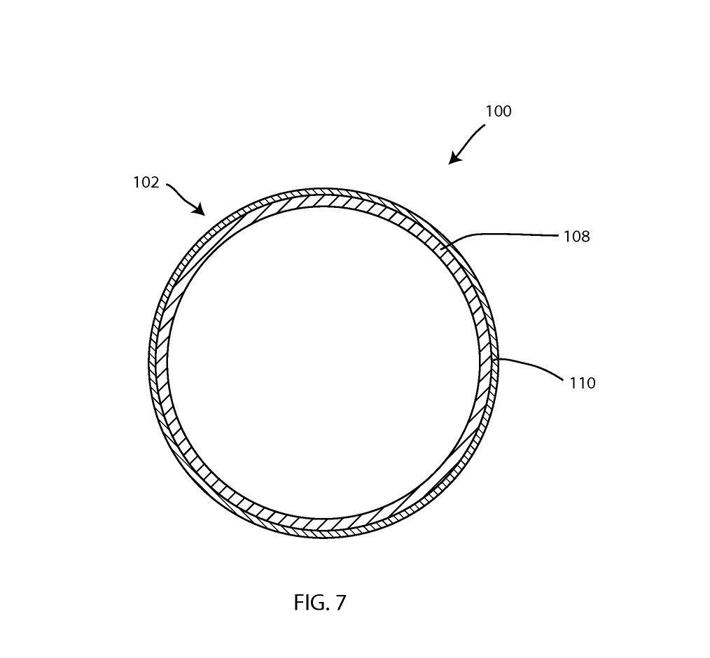

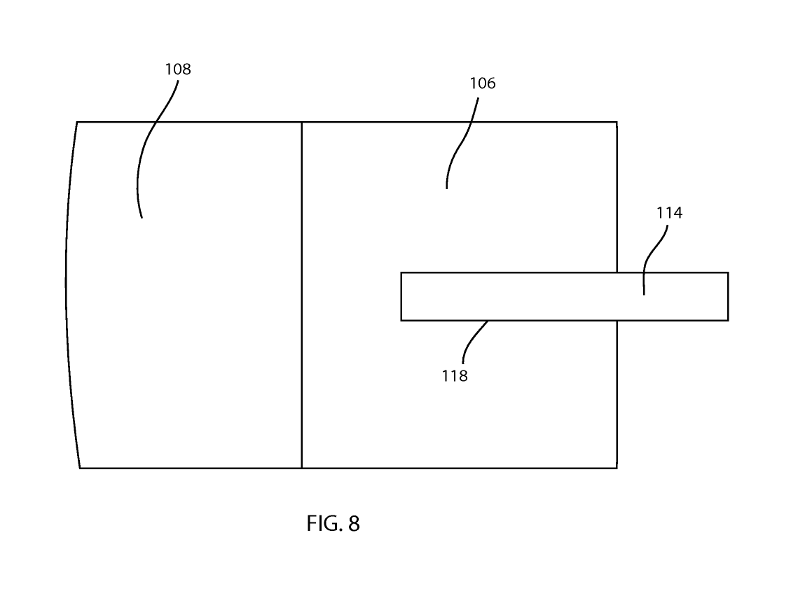

In reference now to the figures, FIG. 1 shows a side view of a coaxial guide coil catheter 100, according to an embodiment of the invention. The coaxial guide coil catheter 100 is configured to be at least partially disposed within a cardiovascular guide catheter. The coaxial guide coil catheter 100 can include a distal portion 102 and a proximal portion 104. The distal portion can comprise a collar 106, an outer jacket 110 which covers a inner coil (see inner coil 108 in FIG. 7, shown relative to outer jacket 110), and a distal tip 112. The proximal portion 104 can comprise a push rod 114. The tip of an obturator (not shown) can typically protrude from the distal tip 112 during placement of the coaxial guide coil catheter within a patient.

The inner coil 108 can comprise metal. The inner coil 108 can be flexible. and can define a first center lumen, such as to allow a therapeutic device to pass through the coaxial guide coil. The inner coil 108 is helically wound in typical embodiments. The inner coil 108 can be a single wire wind, but is more typically a multiple filar winding. In an embodiment, the inner coil 108 comprises 8 or more filar windings. In another embodiment, the inner coil 108 comprises 12 or less filar windings. The ends of the inner coil 108 are welded in some implementations so as to prevent the inner coil from unraveling. In an embodiment, the ends of the inner coil 108 are welded prior to cutting the inner coil to the desired length.

The collar 106 can define a second center lumen. The second center lumen can be consistent with the first center lumen, such that they share a common center axis or a substantially similar diameter. The second center lumen and the first center lumen can be configured to allow a therapeutic device to pass through the coaxial guide coil catheter 100, such as from a position external from a patient's body to a position within the patient's body. In an example, the therapeutic device can be a balloon or a stent. The collar 106 can be, for example, formed of a gold-plated coil.

The push rod 114 can assist a user, such as a surgeon, in inserting, positioning and removal of the coaxial guide coil catheter 100. The push rod 114 can comprise, for example, high tensile stainless steel. The push rod 114 can be semi-rigid, such as to transfer enough force from a user to the rest of the coaxial guide coil catheter 100, such as when position the coaxial guide coil catheter 100.

The coaxial guide coil catheter 100 can comprise radiopaque material 116 at one or more locations, such as the distal tip 112. The radiopaque material 116 can comprise gold. The radiopaque material 116 can be located at known locations along the coaxial guide coil catheter 100, such that a surgeon can use the locations of the radiopaque material to know the positioning of the coaxial guide coil catheter 100, such as with an x-ray.

FIG. 2 shows a bottom view of the coaxial guide coil catheter 100 shown in FIG. 1. The collar 106 can couple the distal portion 102 with the proximal portion 104. The collar can define a notch 118. The notch 118 is configured to receive an end of the push rod 114 in some embodiments, such as to couple the distal portion 102 with the proximal portion 104. The push rod 114 can be attached to the collar 106, such as by welding, brazing, or soldering.

In an example embodiment the coaxial guide coil catheter 100 can have a length of 30 inches or more. The coaxial guide coil catheter 100 can have a length of 80 inches or less. In an embodiment, the coaxial guide coil catheter 100 can have a length of 55 inches. The push rod 114 can have a length of 33 inches or more. The push rod 114 can have a length of 60 inches or less. In an embodiment, the push rod 114 has a length of 43 inches.

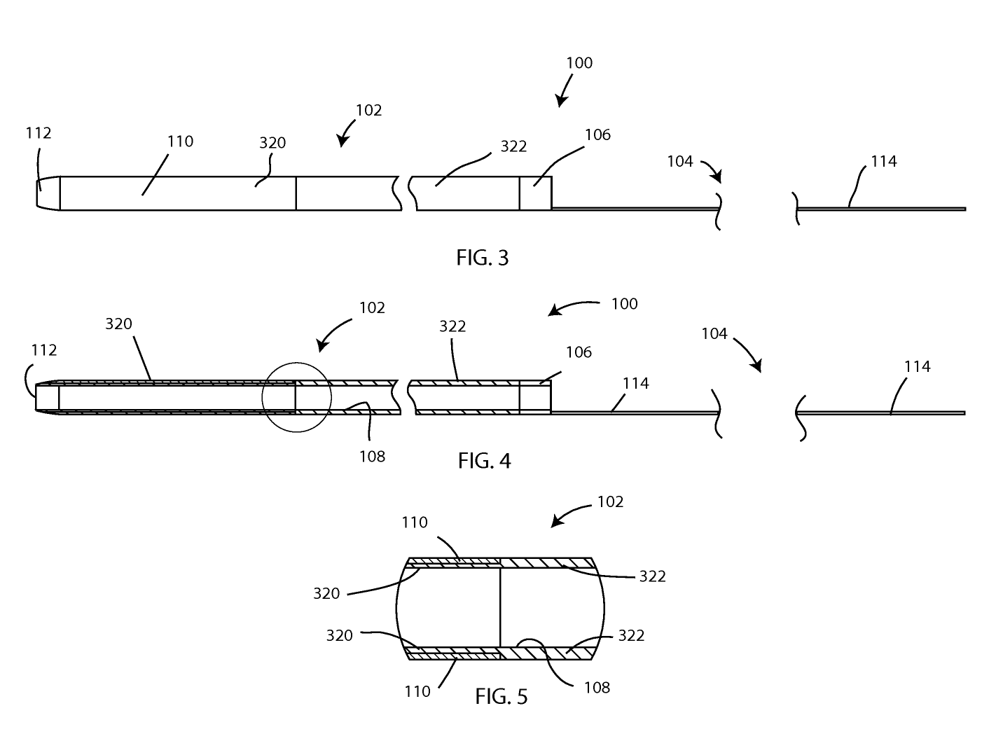

FIG. 3 shows a side view of a coaxial guide coil catheter 100, according to an embodiment of the invention. The distal portion 102 of the coaxial guide coil catheter 100 can comprise a first flexible coil section 320 and a second flexible coil section 322. The outer jacket 110 can cover the outside surface of the first flexible coil section 320. The second flexible coil section 322 can be optionally exposed, such as not being covered by the outer jacket 110. The second flexible coil section 322 can be fluid permeable. In some embodiments the distal tip 112 can be coupled to the first flexible coil section 320, and a collar 106 can be coupled to the second flexible coil section 322.

The outside diameter of the outer jacket 110 can be smaller than the outside diameter of the second flexible coil section 322. In an embodiment, the outside diameter of the outer jacket 110 can be substantially the same size as the outside diameter of the second flexible coil section 322. In an embodiment, the outside diameter of the first flexible coil section 320 can be smaller than the outside diameter of the second flexible coil section 322, such as to allow for the outer jacket 110 to cover the first flexible coil section 320, and the outer jacket 110 can have substantially the same outside diameter as the second flexible coil section 322. The outside diameter of the second flexible coil section 322 can be 1.03 times or more larger than the outside diameter of the first flexible coil section 320. The outside diameter of the second flexible coil section 322 can be 1.1 times or less larger than the outside diameter of the first flexible coil section 320. The first flexible coil section 320 can have an outside diameter of 0.05 inches or more. The first flexible coil section 320 can have an outside diameter of 0.090 inches or less. In an embodiment, the outside diameter of the first flexible coil section 320 can be 0.064 inches. The first flexible coil section 320 can have an inside diameter of 0.047 inches or more. The first flexible coil section 320 can have an inside diameter of 0.084 inches or less. In an embodiment the inside diameter of the first flexible coil section 320 can be 0.057 inches.

The outer jacket 110 can comprise a polymer. The outer jacket 110 can comprise, for example, one or more of the following: Pebax® and urethane. The outside diameter of the outer jacket 110 can be 0.092 inches or less. The outside diameter of the outer jacket 110 can be 0.055 inches or more. In an embodiment the outside diameter for the outer jacket 110 can be 0.084 inches.

FIG. 4 shows a cross-section of the coaxial guide coil catheter 100 shown in FIG. 3. FIG. 5 shows an enlarged portion of FIG. 4 (circled in FIG. 4). The inner coil 108 can have a length of 6 inches or more. The inner coil 108 can have a length of 18 inches or less. In an embodiment, the inner coil 108 can have a length of 12 inches. The inner coil 108 can comprise stainless steel.

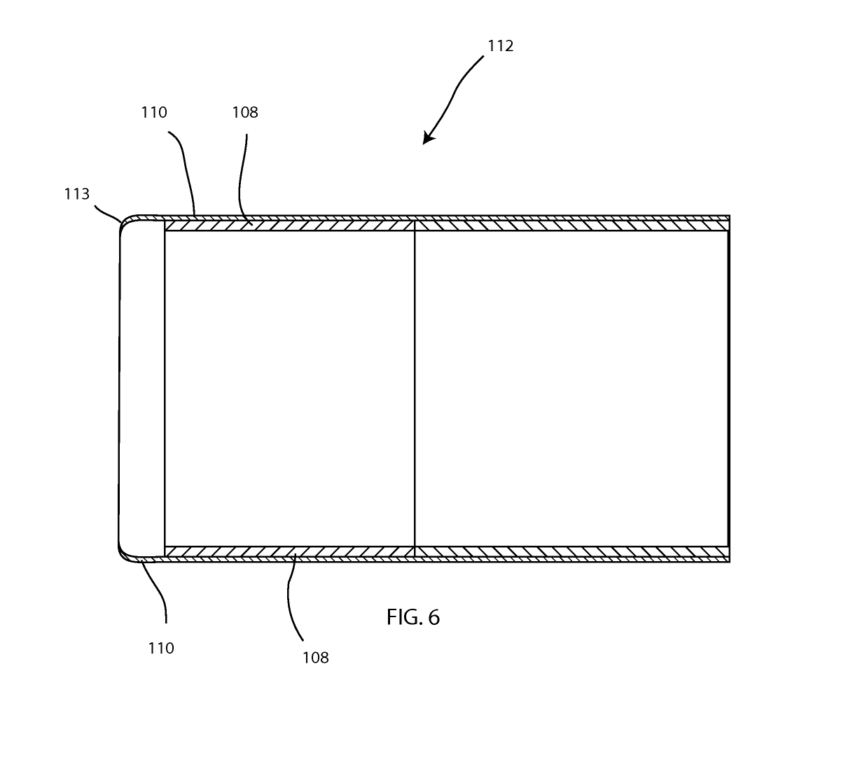

FIG. 6 shows a cross-section view of a distal tip 112 of a coaxial guide coil catheter 100, according to an embodiment. The distal tip 112 can comprise a polymeric material. The polymeric material can be soft relative to the inner coil 108. The distal tip 112 can be softer than one or more of the following: first flexible coil section, the second flexible coil section, and the push rod. The distal tip 112 can comprise radiopaque material, such as to aid a surgeon in knowing where the distal tip is located within a patient. The distal tip 112 can comprises an end 113 with a smaller outside diameter than the outside diameter of the first flexible coil section 320, such as if the distal tip 112 is tapered to enhance the ability for the coaxial coil catheter 100 to be inserted through a catheter or into a portion of a patient's body. In an embodiment, the distal tip 112 comprises an end with an outside diameter substantially the same size as the inside diameter of the first flexible coil section 320. Typically one or more of the following: the distal tip 112, the first flexible coil section 320, and the second flexible coil section 322 can have a circular cross-section.

FIG. 7 shows a cross-section view of a coaxial guide coil catheter 100 taken along line A-A in FIG. 2. The cross section shows the distal portion 102 along with inner coil 108 and outer jacket 110.

FIG. 8 shows a view of the push rod 114 connection with the collar 106, according to an embodiment. The push rod 114 can be attached to the collar 106 by one of the following techniques: welding, brazing, or soldering. The notch 118 defined by the collar 106 can have a length of 0.025 inches or more. The notch 118 defined by the collar 106 can have a length or 0.075 inches or less. In an embodiment, the notch 118 can be 0.05 inches long.

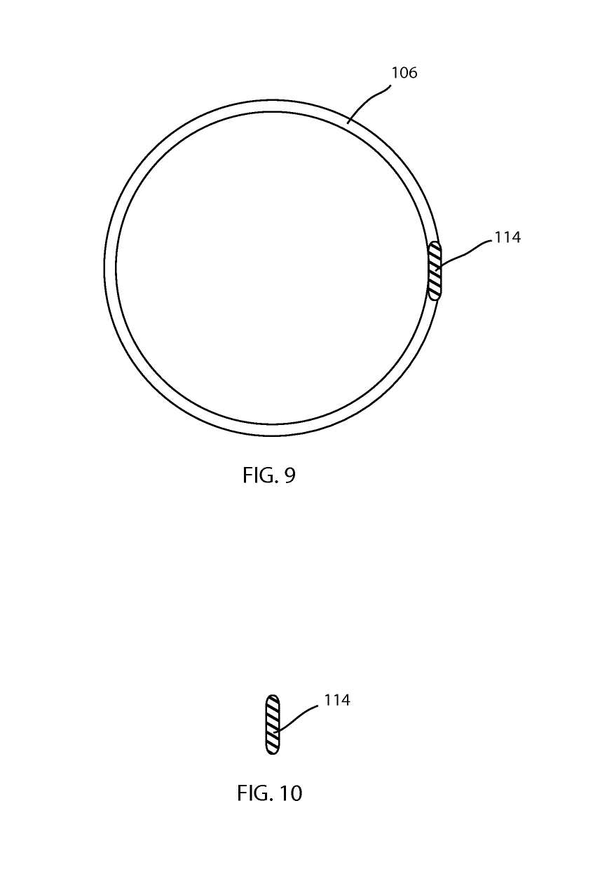

FIG. 9 shows a cross-section view of the connection between the push rod 114 and the collar 106 taken along line B-B in FIG. 2, according to an embodiment. The push rod 114 is typically rectangular can be tangent to the inner diameter of the collar 106.

FIG. 10 shows a cross-section view of a push rod 114 taken along line C-C in FIG. 2, according to an embodiment. In an embodiment, the push rod 114 has a substantially rectangular cross-section. The push rod 114 can have a width of 0.01 inches or more. The push rod 114 can have a width of 0.03 inches or less. In an embodiment, the push rod 114 can have a width of 0.02 inches. The push rod 114 can have a thickness of 0.005 inches or more. The push rod 114 can have a thickness of 0.015 inches or less. In an embodiment, the push rod 114 can have a width of 0.01 inches.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

The claims are:

1. A coaxial guide coil catheter comprising:
a distal portion comprising a collar, an inner coil, and an outer jacket; wherein the inner surface of the inner coil comprises metal and defines a first center lumen; and
a proximal portion comprising a push rod.

2. The coaxial guide coil catheter of any of claims 1 and 3-45, wherein the distal portion comprises a first flexible coil section and a second flexible coil section; and
wherein the outer jacket covers the first flexible coil section of the distal portion.

3. The coaxial guide coil catheter of any of claims 1-2 and 4-45, wherein the collar defines a second center lumen and a notch; wherein the second center lumen is consistent with the first center lumen and the notch is configured to receive a portion of the push rod to couple the distal portion to the proximal portion

4. The coaxial guide coil catheter of any of claims 1-3 and 5-45, wherein the outer diameter of the outer jacket is smaller than the outer diameter of the second flexible coil section.

5. The coaxial guide coil catheter of any of claims 1-4 and 6-45, wherein the outer diameter of the first flexible coil section is smaller than the outer diameter of the second flexible coil section.

6. The coaxial guide coil catheter of any of claims 1-5 and 7-45, wherein coaxial guide coil catheter is configured to be at least partially disposed within a cardiovascular guide catheter.

7. The coaxial guide coil catheter of any of claims 1-6 and 8-45, wherein the push rod comprises high tensile stainless steel.

8. The coaxial guide coil catheter of any of claims 1-7 and 9-45, wherein the push rod is attached to the collar by one of the following techniques: welding, brazing, or soldering.

9. The coaxial guide coil catheter of any of claims 1-8 and 10-45, wherein outer jacket comprises one or more of the following: Pebax® and urethane.

10. The coaxial guide coil catheter of any of claims 1-9 and 11-45, wherein the inner coil is flexible

11. The coaxial guide coil catheter of any of claims 1-10 and 12-45, wherein the inner coil comprises stainless steel.

12. The coaxial guide coil catheter of any of claims 1-11 and 13-45, wherein the outer diameter of the second flexible coil section is 1.03 to 1.1 times larger than the outer diameter of the first flexible coil section

13. The coaxial guide coil catheter of any of claims 1-12 and 14-45, wherein the inner coil comprises multiple rectangular filar windings.

14. The coaxial guide coil catheter of any of claims 1-13 and 15-45, wherein the inner coil comprises 8 to 12 filar windings.

15. The coaxial guide coil catheter of any of claims 1-14 and 16-45, wherein the inner coil comprises a single wire wind.

16. The coaxial guide coil catheter of any of claims 1-15 and 17-45, wherein the distal portion comprises a distal tip.

17. The coaxial guide coil catheter of any of claims 1-16 and 18-45, wherein the distal tip comprises a polymeric material

18. The coaxial guide coil catheter of any of claims 1-17 and 19-45, wherein the polymeric material comprises urethane

19. The coaxial guide coil catheter of any of claims 1-18 and 20-45, wherein a distal tip is coupled to the first flexible coil section.

20. The coaxial guide coil catheter of any of claims 1-19 and 21-45, wherein the distal tip comprises a radiopaque material.

21. The coaxial guide coil catheter of any of claims 1-20 and 22-45, wherein the radiopaque material comprises gold.

22. The coaxial guide coil catheter of any of claims 1-21 and 23-45, wherein the first and second center lumens are configured to allow a therapeutic device to pass through.

23. The coaxial guide coil catheter of any of claims 1-22 and 24-45, wherein the therapeutic device comprises a stent or a balloon.

24. The coaxial guide coil catheter of any of claims 1-23 and 25-45, wherein the distal tip is softer than the first flexible coil section.

25. The coaxial guide coil catheter of any of claims 1-24 and 26-45, wherein the distal tip has a circular cross-section.

26. The coaxial guide coil catheter of any of claims 1-25 and 27-45, wherein the inner coil is helically wound.

27. The coaxial guide coil catheter of any of claims 1-26 and 28-45, wherein the first flexible coil section has a circular cross-section.

28. The coaxial guide coil catheter of any of claims 1-27 and 29-45, wherein the second flexible coil section has a circular cross-section.

29. The coaxial guide coil catheter of any of claims 1-28 and 30-45, wherein the second flexible coil section is fluid permeable.

30. The coaxial guide coil catheter of any of claims 1-29 and 31-45, wherein the push rod is semi-rigid.

31. The coaxial guide coil catheter of any of claims 1-30 and 32-45, wherein the inner coil comprises one or more welds.

32. The coaxial guide coil catheter of any of claims 1-31 and 33-45, wherein the inner coil comprises welded ends.

33. The coaxial guide coil catheter of any of claims 1-32 and 34-45, wherein an inner surface of the push rod is tangent with the inner diameter of the collar.

34. The coaxial guide coil catheter of any of claims 1-33 and 35-45, wherein the distal tip comprises an end with a smaller outside diameter than the outside diameter of the first flexible coil section.

35. The coaxial guide coil catheter of any of claims 1-34 and 36-45, wherein the distal tip comprises an end with an outside diameter substantially the same size as the inside diameter of the first flexible coil section.

36. The coaxial guide coil catheter of any of claims 1-35 and 37-45, having a length of between 30 inches and 80 inches.

37. The coaxial guide coil catheter of any of claims 1-36 and 38-45, wherein push rod has a length of between 33 inches and 53 inches.

38. The coaxial guide coil catheter of any of claims 1-37 and 39-45, wherein the inner coil has a length of between 4 inches and 24 inches.

39. The coaxial guide coil catheter of any of claims 1-38 and 40-45, wherein the outside diameter of the first flexible coil section is between 0.05 inches and 0.090 inches.

40. The coaxial guide coil catheter of any of claims 1-39 and 41-45, wherein the inside diameter of the first flexible coil section is between 0.047 inches and 0.084.

41. The coaxial guide coil catheter of any of claims 1-40 and 42-45, wherein the portion of the push rod within the notch in the collar is between 0.075 inches and 0.25 inches long.

42. The coaxial guide coil catheter of any of claims 1-41 and 43-45, wherein the push rod has a width of between 0.01 inches and 0.03 inches.

43. The coaxial guide coil catheter of any of claims 1-42 and 44-45, wherein the push rod has a thickness of between 0.005 inches and 0.015 inches.

44. The coaxial guide coil catheter of any of claims 1-43 and 45, wherein the outer diameter of the outer jacket is between 0.055 inches and 0.092 inches.

45. The coaxial guide coil catheter of any of claims 1-44, wherein the outer diameter of the outer jacket is substantially the same size as the outer diameter of the second flexible coil section.

46. A coaxial guide coil catheter comprising:
a distal portion comprising a collar, an inner coil, and an outer jacket; wherein the inner coil defines a first center lumen;
a proximal portion comprising a push rod; and
the outer jacket comprises a polymer;
wherein the collar defines a notch configured to receive a portion of the push rod to couple the distal portion to the proximal portion.

47. The coaxial guide coil catheter of any of claims 46 and 48-66, wherein the distal portion comprises a first flexible coil section and a second flexible coil section;
and wherein the outer jacket covers the first flexible coil section of the distal portion.

48. The coaxial guide coil catheter of any of claims 46-47 and 49-66, wherein the collar defines a second center lumen and a notch; wherein the second center lumen is consistent with the first center lumen and the notch is configured to receive a portion of the push rod to couple the distal portion to the proximal portion

48. The coaxial guide coil catheter of any of claims 46-48 and 50-66, wherein the outer diameter of the outer jacket is smaller than the outer diameter of the second flexible coil section.

49. The coaxial guide coil catheter of any of claims 46-49 and 51-66, wherein the outer diameter of the first flexible coil section is smaller than the outer diameter of the second flexible coil section.

50. The coaxial guide coil catheter of any of claims 46-50 and 52-66, wherein coaxial guide coil catheter is configured to be at least partially disposed within a cardiovascular guide catheter.

51. The coaxial guide coil catheter of any of claims 46-51 and 53-66, wherein the push rod comprises high tensile stainless steel.

52. The coaxial guide coil catheter of any of claims 46-52 and 54-66, wherein the push rod is attached to the collar by one of the following techniques: welding, brazing, or soldering.

53. The coaxial guide coil catheter of any of claims 46-53 and 55-66, wherein outer jacket comprises one or more of the following: Pebax® and urethane.

54. The coaxial guide coil catheter of any of claims 46-54 and 56-66, wherein the inner coil is flexible

55. The coaxial guide coil catheter of any of claims 46-55 and 57-66, wherein the inner coil comprises stainless steel.

56. The coaxial guide coil catheter of any of claims 46-56 and 58-66, wherein the outer diameter of the second flexible coil section is 1.03 to 1.1 times larger than the outer diameter of the first flexible coil section

57. The coaxial guide coil catheter of any of claims 46-57 and 59-66, wherein the inner coil comprises multiple filar windings.

58. The coaxial guide coil catheter of any of claims 46-58 and 60-66, wherein the inner coil comprises 8 to 12 filar windings.

59. The coaxial guide coil catheter of any of claims 46-59 and 61-66, wherein the inner coil comprises a single wire wind.

60. The coaxial guide coil catheter of any of claims 46-60 and 62-66, wherein the distal portion comprises a distal tip.

61. The coaxial guide coil catheter of any of claims 46-61 and 63-66, wherein the distal tip comprises a polymeric material

62. The coaxial guide coil catheter of any of claims 46-62 and 64-66, wherein the polymeric material comprises urethane

63. The coaxial guide coil catheter of any of claims 46-63 and 65-66, wherein a distal tip is coupled to the first flexible coil section.

64. The coaxial guide coil catheter of any of claims 46-64 and 66, wherein the distal tip comprises a radiopaque material.

65. The coaxial guide coil catheter of any of claims 46-65, wherein the radiopaque material comprises gold.

66. A coaxial guide coil catheter comprising:
a distal portion comprising a collar, an inner coil, and an outer jacket; wherein the inner coil defines a first center lumen; and
a proximal portion comprising a push rod;
wherein the collar defines a second center lumen and a notch; wherein the second center lumen is consistent with the first center lumen and the a notch is configured to receive a portion of the push rod to couple the distal portion to the proximal portion;
wherein the inner coil comprises a first end and a second end, and the first and second ends comprise at least one weld.

67. The coaxial guide coil catheter of any of claims 67 and 69-87, wherein the distal portion comprises a first flexible coil section and a second flexible coil section;
and wherein the outer jacket covers the first flexible coil section of the distal portion.

68. The coaxial guide coil catheter of any of claims 67-68 and 70-87, wherein the collar defines a second center lumen and a notch; wherein the second center lumen is consistent with the first center lumen and the notch is configured to receive a portion of the push rod to couple the distal portion to the proximal portion

69. The coaxial guide coil catheter of any of claims 67-69 and 71-87, wherein the outer diameter of the outer jacket is smaller than the outer diameter of the second flexible coil section.

70. The coaxial guide coil catheter of any of claims 67-70 and 72-87, wherein the outer diameter of the first flexible coil section is smaller than the outer diameter of the second flexible coil section.

71. The coaxial guide coil catheter of any of claims 67-71 and 73-87, wherein coaxial guide coil catheter is configured to be at least partially disposed within a cardiovascular guide catheter.

72. The coaxial guide coil catheter of any of claims 67-72 and 74-87, wherein the push rod comprises high tensile stainless steel.

73. The coaxial guide coil catheter of any of claims 67-73 and 75-87, wherein the push rod is attached to the collar by one of the following techniques: welding, brazing, or soldering.

74. The coaxial guide coil catheter of any of claims 67-74 and 76-87, wherein outer jacket comprises one or more of the following: Pebax® and urethane.

75. The coaxial guide coil catheter of any of claims 67-75 and 77-87, wherein the inner coil is flexible

76. The coaxial guide coil catheter of any of claims 67-76 and 78-87, wherein the inner coil comprises stainless steel.

78. The coaxial guide coil catheter of any of claims 67-77 and 79-87, wherein the outer diameter of the second flexible coil section is 1.03 to 1.1 times larger than the outer diameter of the first flexible coil section

79. The coaxial guide coil catheter of any of claims 67-78 and 80-87, wherein the inner coil comprises multiple filar windings.

80. The coaxial guide coil catheter of any of claims 67-79 and 81-87, wherein the inner coil comprises 8 to 12 filar windings.

81. The coaxial guide coil catheter of any of claims 67-80 and 82-87, wherein the inner coil comprises a single wire wind.

82. The coaxial guide coil catheter of any of claims 67-81 and 83-87, wherein the distal portion comprises a distal tip.

83. The coaxial guide coil catheter of any of claims 67-82 and 84-87, wherein the distal tip comprises a polymeric material

84. The coaxial guide coil catheter of any of claims 67-83 and 85-87, wherein the polymeric material comprises urethane

85. The coaxial guide coil catheter of any of claims 67-84 and 86-87, wherein a distal tip is coupled to the first flexible coil section.

86. The coaxial guide coil catheter of any of claims 67-85 and 87, wherein the distal tip comprises a radiopaque material.

87. The coaxial guide coil catheter of any of claims 67-86, wherein the radiopaque material comprises gold.

88. A coaxial guide coil catheter comprising:
a distal portion comprising a collar, an inner coil, and an outer jacket; wherein the inner coil comprises metal and defines a first center lumen; and
a proximal portion comprising a push rod;
wherein the collar defines a second center lumen and a notch; wherein the second center lumen is consistent with the first center lumen and the notch is configured to receive a portion of the push rod to couple the distal portion to the proximal portion.

89. The coaxial guide coil catheter of any of claims 88 and 90-108, wherein the distal portion comprises a first flexible coil section and a second flexible coil section; and wherein the outer jacket covers the first flexible coil section of the distal portion.

90. The coaxial guide coil catheter of any of claims 88-89 and 91-108, wherein the collar defines a second center lumen and a notch; wherein the second center lumen is consistent with the first center lumen and the notch is configured to receive a portion of the push rod to couple the distal portion to the proximal portion

91. The coaxial guide coil catheter of any of claims 88-90 and 92-108, wherein the outer diameter of the outer jacket is smaller than the outer diameter of the second flexible coil section.

92. The coaxial guide coil catheter of any of claims 88-91 and 93-108, wherein the outer diameter of the first flexible coil section is smaller than the outer diameter of the second flexible coil section.

93. The coaxial guide coil catheter of any of claims 88-92 and 94-108, wherein coaxial guide coil catheter is configured to be at least partially disposed within a cardiovascular guide catheter.

94. The coaxial guide coil catheter of any of claims 88-93 and 95-108, wherein the push rod comprises high tensile stainless steel.

95. The coaxial guide coil catheter of any of claims 88-94 and 96-108, wherein the push rod is attached to the collar by one of the following techniques: welding, brazing, or soldering.

96. The coaxial guide coil catheter of any of claims 88-95 and 97-108, wherein outer jacket comprises one or more of the following: Pebax® and urethane.

97. The coaxial guide coil catheter of any of claims 88-96 and 98-108, wherein the inner coil is flexible

98. The coaxial guide coil catheter of any of claims 88-97 and 99-108, wherein the inner coil comprises stainless steel.

99. The coaxial guide coil catheter of any of claims 88-98 and 100-108, wherein the outer diameter of the second flexible coil section is 1.03 to 1.1 times larger than the outer diameter of the first flexible coil section

100. The coaxial guide coil catheter of any of claims 88-99 and 101-108, wherein the inner coil comprises multiple filar windings.

101. The coaxial guide coil catheter of any of claims 88-100 and 102-108, wherein the inner coil comprises 8 to 12 filar windings.

102. The coaxial guide coil catheter of any of claims 88-101 and 103-108, wherein the inner coil comprises a single wire wind.

103. The coaxial guide coil catheter of any of claims 88-102 and 104-108, wherein the distal portion comprises a distal tip.

104. The coaxial guide coil catheter of any of claims 88-103 and 105-108, wherein the distal tip comprises a polymeric material

105. The coaxial guide coil catheter of any of claims 88-104 and 106-108, wherein the polymeric material comprises urethane

106. The coaxial guide coil catheter of any of claims 88-105 and 107-108, wherein a distal tip is coupled to the first flexible coil section.

107. The coaxial guide coil catheter of any of claims 88-106 and 108, wherein the distal tip comprises a radiopaque material.

108. The coaxial guide coil catheter of any of claims 88-107, wherein the radiopaque material comprises gold.
